(19) United States
(12) Patent Application Publication
(10) Pub. No.: US 2003/0087231 A1
(43) Pub. Date: May 8, 2003

(54) Methods and compositions for preparation of a polynucleotide array

(76) Inventors: Donna G. Albertson, Lafayette, CA (US); Daniel Pinkel, Walnut Creek, CA (US); Antoine Snijders, San Francisco, CA (US)

Correspondence Address: QUINE INTELLECTUAL PROPERTY LAW GROUP, P.C., P O BOX 458, ALAMEDA, CA, 94501, US

(21) Appl. No.: 09/574,386

(22) Filed: May 19, 2000

(51) Int. Cl. C12Q 1/68; C12M 1/34; B05D 3/00

(52) U.S. Cl. 435/6; 435/287.2; 427/2.11

(57) ABSTRACT

The present invention provides an amplification method for preparing target solutions for polynucleotide arrays. This method produces amplification products that can be used to make relatively low-viscosity target solutions that are representative of the starting polynucleotides, which facilitates array fabrication by robotic spotting. Other aspects of the invention include target solutions, methods of forming arrays from such solutions, and the arrays so produced.

This invention was made with Government support under Grant Nos. CA80314 and CA83040, awarded by the National Institutes of Health. The Government has certain rights in this invention.

### BACKGROUND OF THE INVENTION

1. Field of the Invention

The present invention relates to methods and compositions for fabricating polynucleotide arrays. More particularly, the invention relates to methods that render high molecular weight DNA suitable for robotic spotting.

2. Description of the Related Art

Array-based technology has been used to advantage in genomic mapping, “fingerprinting” of polynucleotides, DNA sequencing, analysis of genomic copy number, and expression monitoring. Arrays employed in such studies typically consist of a matrix of polynucleotides immobilized on a substrate at distinct locations. Hybridization of the array with a sample of labeled polynucleotides, followed by signal detection at each location, allows the simultaneous analysis of a large number of hybridization interactions in one procedure.

A variety of methods are currently available for making polynucleotide arrays on substrates. In an early example of this approach, a vacuum manifold is used to transfer aqueous samples of DNA from a microtiter plate to a porous membrane to produce a “dot blot.” A common variant of this procedure is a “slot-blot” method in which the wells have highly-elongated oval shapes. The DNA is immobilized on the porous membrane by baking the membrane or exposing it to UV radiation. This is a manual procedure practical for making one array at a time and usually limited to 96 samples per array. “Dot-blot” procedures are therefore inadequate for applications in which many samples must be analyzed.

An alternate method of creating ordered arrays of polynucleotide sequences involves synthesizing different polynucleotide sequences at different discrete regions of a substrate. This method relies on elaborate synthetic schemes and is therefore generally used only for fabricating arrays of relatively short polynucleotides.

A technique more suitable for making ordered arrays of longer polynucleotides uses a sample dispenser mounted on a device that can be precisely positioned to spot samples onto a substrate. For example, U.S. Pat. No. 5,807,522 (issued Sep. 15, 1998 to Brown and Shalon) describes a device that facilitates mass fabrication of microarrays characterized by a large number of micro-sized assay regions separated by a distance of 50-200 microns or less and a well-defined amount of analyte (typically in the picomolar range) associated with each region of the array.

An alternative approach to robotic spotting uses an array of pins or capillary dispensers dipped into the wells, e.g., the 96 wells of a microtiter plate, for transferring an array of samples to a substrate. Arrays can also be fabricated by coating elements such as beads or optical fibers with samples to form target elements. U.S. Pat. No. 5,830,645 (issued Nov. 3, 1998 to Pinkel et al.) describes the use of beads to produce a polynucleotide array, and U.S. Pat. No. 5,690,894 (issued on Nov. 25, 1997 to Pinkel et al.) discloses a polynucleotide array fabricated from optical fibers.

While these conventional techniques are suitable for producing arrays of relatively low molecular weight polynucleotides, the arraying of a large number of high molecular weight polynucleotides, such as yeast artificial chromosome (YAC), bacterial artificial chromosomes (BAC), P1, or PAC clones, presents unique challenges. For many applications, for example, it may be desirable to make arrays having on the order of 15,000-30,000 polynucleotides of up to about a megabase in complexity. Dot and slot blot techniques are impractical for fabricating such large arrays and cannot be used to make microarrays, which often have distinct polynucleotide regions separated by a hundred microns or less. Conventional synthetic techniques are unsatisfactory for producing arrays of high molecular weight polynucleotides due to the practical limitations of synthetic methods. Robotic spotting techniques have suffered from the difficulties associated with spotting the highly viscous solutions of high molecular weight polynucleotides. The preparation of arrays from polynucleotides derived from single-copy vectors, such as YACs, BACS, P1s, and PACs, is further complicated by the difficulty of preparing sufficient quantities of DNA for arraying.

### SUMMARY OF THE INVENTION

The present invention provides methods for making target solutions and polynucleotide arrays that overcome the deficiencies of conventional techniques, facilitating the production of polynucleotide arrays with target elements containing polynucleotides that are representative of a collection of polynucleotides of interest.

More specifically, the invention includes a method for preparing amplification products from samples of double-stranded polynucleotide fragments, each derived from a starting polynucleotide, as templates for ligation-mediated PCR. Preferably, the samples of double-stranded polynucleotide fragments are obtained using one or more restriction endonucleases. Adapters are ligated to each end of the polynucleotide fragments to produce modified polynucleotide fragments. Each adapter includes a first strand and a second strand, and the second strand has a region of substantial complementarity to a region of the first strand. The modified polynucleotide fragments are then amplified to produce an amplification product for each sample of polynucleotide fragments. Each amplification product is isolated and resuspended to form a target solution suitable for application to a substrate to produce an array of polynucleotides.

The invention also includes a collection of target solutions prepared using the above amplification method. Preferred target solutions include dimethyl sulfoxide at a concentration of about 20% by volume.

In one embodiment, the double-stranded polynucleotide fragments are derived from a polynucleotide library, which is preferably a genomic DNA library or a cDNA library. As the methods of the invention are particularly useful for arraying high molecular weight polynucleotides (e.g., those having a complexity of greater than 50 kilobases), the double-stranded polynucleotide fragments can be derived from YAC, BAC, P1 or PAC clones.

The invention also provides a method for producing a polynucleotide array in which the target solutions of the invention are applied to one or more substrates. In one embodiment, each target solution is applied to a distinct location on one substrate. In another embodiment, target solutions are applied to different substrates, such as beads or optical fibers, to produce target elements. These two fabrication techniques can be used in combination, if desired. In a preferred embodiment, the target solutions are robotically spotted on the substrate.

Also within the scope of the invention is a polynucleotide array produced according to the above-described methods that is representative of a collection of starting polynucleotides and includes at least 100 amplification products in a 1 cm² region of substrate.

### BRIEF DESCRIPTION OF THE DRAWINGS

FIG. 1 shows the results of comparative genomic hybridization (“CGH”) of DNA from the breast cancer cell line BT474 (labeled with FITC-dCTP) and normal female DNA (labeled with Cy3-dCTP) to an array containing target elements prepared from BAC clones containing chromosome 20 sequences using the methods of the invention. The ratio of the BT474 DNA:normal DNA hybridization signal (normalized ratio) is shown for amplification products prepared from BAC clones using ligation-mediated PCR (PCR1-3), as compared to historical data from an array of BAC DNA that was isolated conventionally. Three independently prepared amplification products were produced for most of the BAC clones that were amplified. These results demonstrate that ligation-mediated PCR produces an amplification product that is highly representative of (i.e., performs equivalently to) the BAC clone that serves as the template.

FIG. 2 shows the results of CGH of DNA from the breast cancer cell line BT474 (labeled with FITC-dCTP) and normal female DNA (labeled with Cy3-dCTP) to an array containing target elements prepared by ligation-mediated PCR from about 400 BAC clones that sample the human genome. Each bar represents the hybridization signal ratio obtained for a clone, and the clones are grouped by order on each chromosome. Chromosome numbers are indicated on the X-axis. Panel A illustrates that, as expected, the ratio of the hybridization signal for two samples of normal female DNA is essentially constant for all targets. The results in panel A are normalized to about 1.0. Panel B shows the (non-normalized) ratios of the signals observed for the BT474:normal DNA hybridization and indicates that copy number variations in BT474 DNA, especially those present on chromosome 20, are readily detectable in this system.

### DETAILED DESCRIPTION OF THE INVENTION

The present invention provides a method for preparing target solutions for polynucleotide arrays by amplification of the polynucleotides to be arrayed. This procedure produces large quantities of amplification products that can be used to make relatively low-viscosity target solutions that are representative of the starting polynucleotides, which facilitates array fabrication by robotic spotting.

### Definitions

The term “array” refers to a collection of elements, wherein each element is uniquely identifiable. For example, the term can refer to a substrate bearing an arrangement of elements, such that each element has a physical location on the surface of the substrate that is distinct from the location of every other element. In such an array, each element can be identifiable simply by virture of its location. Typical arrays of this type include elements arranged linearly or in a two-dimensional matrix, although the term “array” encompasses any configuration of elements and includes elements arranged on non-planar, as well as planar, surfaces. Non-planar arrays can be made, for example, by arranging beads, pins, or fibers to form an array. The term “array” also encompasses collections of elements that do not have a fixed relationship to one another. For example, a collection of beads in which each bead has an identifying characteristic can constitute an array.

The elements of an array are termed “target elements.”

As used herein with reference to target elements, the term “distinct location” means that each element is physically separated from every other target element such that a signal (e.g., a fluorescent signal) from a labeled molecule bound to target element can be uniquely attributed to binding at that target element.

A “microarry” is an array in which the density of the target elements on the substrate surface is at least about 100/cm².

The term “polynucleotide” refers to a deoxyribonucleotide or ribonucleotide polymer in either single- or double-stranded form, and unless otherwise limited, would encompass known analogs of natural nucleotides that can function in a similar manner to naturally occurring nucleotides.

A polynucleotide whose sequences are to be included in a single target element in a polynucleotide array is termed a “starting polynucleotide.”

The method of the invention produces a “polynucleotide product” that is representative of the starting polynucleotide.

A polynucleotide product is said to be “representative” of a starting polynucleotide if the hybridization signal observed from the polynucleotide product is sufficiently similar to that observed from the starting polynucleotide that the polynucleotide product can be substituted for the starting polynucleotide in a hybridization assay. In other words, a representative polynucleotide product performs essentially equivalently to the starting polynucleotide in a hybridization assay of interest. An array of polynucleotides is said to be “representative” of a collection of starting polynucleotides if the polynucleotides present in each target element are representative of the corresponding starting polynucleotide.

A polynucleotide is “double-stranded” if it contains two polynucleotide strands joined by hydrogen bonding. The polynucleotide strands need not be coextensive (i.e, a double-stranded polynucleotide need not be double-stranded along the entire length of both strands).

A “polynucleotide library” is a collection of polynucleotides derived, directly or indirectly, from a biological sample. Typical polynucleotide libraries include cloning vectors containing inserts corresponding to polynucleotide sequences in a biological sample; however, the term “polynucleotide library” also includes collections of polynucleotides that are not present in cloning vectors, such as, for example, genomic DNA, cDNA synthesized from mRNA, or polynucleotides amplified from a sample.

The term “adapter” is used herein to refer to a double-stranded polynucleotide that can be ligated to the end of a polynucleotide fragment to facilitate ligation-mediated amplification. Adapters are usually (but not necessarily) oligonucleotides of less than 100 bases in length.

“5′ or 3′ extensions” are single-stranded extensions at either end (or both ends) of an otherwise double-stranded polynucleotide. Typically, such extensions are produced upon digestion with a restriction endonuclease, but the invention is not limited to 5′ or 3′ extensions produced in this manner. Such extensions are said to be “common” if they share sufficient sequence homology to hybridize to a given oligonucleotide. For convenience, the method of the invention generally employs polynucleotide fragments that have 5′ extensions that share the identical sequence.

The term “complexity” is used herein according to standard meaning of this term as established by Britten et al. (1974) Methods of Enzymol. 29:363. See also, Cantor and Schimmel Biophysical Chemistry: Part III at 1228-1230 for a further explanation of nucleic acid complexity.

As used herein, the term “substantially complementary” describes sequences that are sufficiently complementary to one another to allow for specific hybridization under appropriately stringent hybridization conditions. “Specific hybridization” refers to the binding of a polynucleotide to a target nucleotide sequence in the absence of substantial binding to other nucleotide sequences present in the hybridization mixture under defined stringency conditions. Those of skill in the art recognize that relaxing the stringency of the hybridizing conditions allows sequence mismatches to be tolerated.

### Preparation of Target Solutions

The invention provides methods for preparing target solutions, as well as target solutions suitable for preparing a polynucleotide array that is representative of the collection of starting polynucleotides from which the target solutions are derived.

Any type of polynucleotide can be employed as the starting polynucleotide in the methods of the invention. Typically, the starting polynucleotide is a DNA molecule, which can be obtained by any available means. The polynucleotide can a have sequence corresponding to a natural polynucleotide sequence found in any organism, preferably a mammal, and more preferably a human. Alternatively, the polynucleotide sequence can be one that is not present in nature.

In preferred embodiments, each of the starting polynucleotides is derived from a defined region of the genome (for example, a clone or several contiguous clones from a genomic library) or corresponds to an expressed sequence (for example, a full-length or partial cDNA). The polynucleotides can also comprise amplification products, such as inter-Alu or degenerate oligonucleotide primer PCR products derived from such clones or from sample polynucleotides.

For arrays designed to analyze copy number variations in, for example, genomic DNA from tumor cells, the starting polynucleotides are derived from specific genes or chromosomal regions that are being tested for increased or decreased copy number in cells of interest. Such arrays can be used in methods such as Comparative Genomic Hybridization (CGH). For arrays designed to analyze gene expression, the starting polynucleotides are generally full-length or partial cDNAs. In a variation of this embodiment, the polynucleotides are full-length or partial cDNAs corresponding to expressed sequences that are suspected of being transcribed at abnormal levels.

Polynucleotides of unknown significance or location in the genome can also be employed in the methods of the invention. An array of such polynucleotides could represent locations that sample, either continuously or at discrete points, any desired portion of a genome, including, but not limited to, an entire genome, a single chromosome, or a portion of a chromosome. The number of polynucleotide elements in the array and the complexity of the polynucleotides would determine the density of sampling. For example, an array of 300 elements, each element containing DNA from a different genomic clone, could sample the entire human genome at 10 megabase (Mb) intervals. An array of 30,000 elements, each containing 100 kb of genomic DNA could give complete coverage of the human genome. Similarly, an array of polynucleotides derived from uncharacterized cDNA clones would permit identification of those that are differentially expressed in different cell types or under different culture conditions.

In preferred embodiments, the starting polynucleotides are derived from a polynucleotide library. The polynucleotide library can be a genomic DNA library, a cDNA library, or simply a collection of genomic or cDNA molecules or polynucleotides amplified from a sample. Although libraries using any type of cloning vector, such as eukaryotic (e.g., yeast), procaryotic, or viral vectors, can be employed in the methods of the invention, the methods are particularly useful for producing target solutions from YAC, BAC, P1 , PAC or cosmid libraries. YAC, BAC, P1, and PAC vectors are designed to accommodate very large (i.e., up to several hundred kb) inserts, and thus clones from such libraries are difficult to array using conventional methods for array fabrication.

For most applications, the starting polynucleotides each have a complexity of at least about 1 kb, although this is not a requirement. In specific embodiments, the starting polynucleotides each have a complexity of at least about 5, 10, 20, 30, 40, and 50 kb, and more preferably at least about 100, 200, 300, 400, and 500 kb. For most applications, the complexity is less than about 1.1 Mb but the methods of the invention can be applied to higher complexity polynucleotides, if desired.

Ligation-Mediated Amplification of Polynucleotides for Target Solutions

In one embodiment, the target solutions are prepared using a ligation-mediated amplification procedure described by Klein, C. A., et al. (1999) Proc. Natl. Acad. Sci. USA 96:4494-4499 for global amplification of DNA from single eukaryotic cells. Ligation-mediated PCR requires double-stranded polynucleotide fragments, preferably having 5′ or 3′ extensions. Adapters are ligated to each end of the polynucleotide fragments, which provides the fragments with common priming sites for amplification. Adapters are typically designed to serve as efficient amplification primers so that unligated strands of the adapters can be employed to amplify the sequences between the priming sites. This approach allows amplification of any polynucleotide without prior knowledge of the nucleotide sequence and allows the production of amplification products that are representative of the starting polynucleotide used as the amplification template.

The starting material for amplifying polynucleotides for target solutions of the invention is a plurality of samples of double-stranded polynucleotide fragments. Each sample of polynucleotide fragments is derived from a starting polynucleotide, i.e., one whose sequences are to be included at a distinct location in the array. The starting polynucleotides are obtained by any standard procedure that produces polynucleotides sufficiently free of contaminants to allow the generation of polynucleotide fragments that can be amplified. Where the starting polynucleotide is a recombinant clone, for example, the polynucleotide is preferably substantially free of host cell DNA and non-polynucleotide contaminants. Example 1 describes the isolation of BAC clones for arraying by standard alkaline lysis.

Blunt-ended fragments can be employed in ligation-mediated amplification, but fragments having common 5′ or 3′ extensions are preferred. Double-stranded polynucleotide fragments with 5′ or 3′ extensions are most conveniently obtained by digesting each starting polynucleotide with a restriction endonuclease that produces such fragments. A large number of restriction enzymes are available, and many suitable for use in the claimed method are described in Sambrook et al. (1989) Molecular Cloning: A Laboratory Manual, 2nd Edition (Cold Spring Harbor Laboratory Press).

The restriction enzyme employed preferably has a cutting frequency such that it is expected to produce polynucleotide fragments that are small enough to allow amplification using standard techniques. Preferably, polynucleotide fragments having an average length of less than about 5 kilobases (kb), more preferably less than about 2 kb, are generated for use in the method of the invention. Typically, the average length of such polynucleotide fragments is greater than about 50 basepairs (bp). The cutting frequencies of the available restriction enzymes can be determined statistically to identify restriction enzymes that produce fragments in this range of sizes. If a given restriction enzyme has too few or too many cutting sites in a polynucleotide, the selection of an alternate enzyme (or an additional enzyme, in the case of too few cutting sites) is within the level of skill in the art. Restriction enzymes used for ligation mediated PCR typically have at least 4-base cleavage sites, and preferably 4-, 5-, or 6-base cleavage sites. Examples of suitable restriction enzymes include the following 4-base cutters: CviJI, MnlI, AluI, BsuFI, HapII, HpaII, MseI, MspI, AccII, BstUI, BsuEI, FnuDII, ThaI, Bce243I, BsaPI, Bsp67I, BspAI,BspPII, BsrPII, BssGII, BstEIII, BstXII, CpaI, CviAI, DpnII, FnuAII, FnuCI, FnuEI, MboI, MmeII, MnoIII, MosI, MthI, NdeII, NflI, NlaII, NsiAI, NsuI, PfaI, Sau3AI, SinMI, HhaI, HinPI, BsuRI, HaeIII, NgoII, CviQI, RsaI, TaqI, and TthHBI.

More than one restriction endonuclease can be employed, if desired. Depending on the combination of restriction enzymes, an additional primer(s) may be required to ensure that all fragments are amplified to produce an amplification product that is representative of the starting polynucleotide.

Restriction digests are carried out under standard conditions, usually those recommended by the manufacturer.

After obtaining samples of double-stranded polynucleotide fragments corresponding to each starting polynucleotide, adapters are added to each end of the polynucleotide fragments to produce modified polynucleotide fragments. The considerations for designing adapters suitable for use in the present invention do not differ from those in standard ligation-mediated amplification procedures. See, e.g., Klein, C. A., et al. (1999) Proc. Natl. Acad. Sci. USA 96:4494-4499; Smith, D. R. (1992) PCR Methods and Applications 2:21-27.

In particular, adapters contain two polynucleotide strands, one or both of which is/are capable of serving as amplification primers. The second strand has a first region of substantial complementarity to a first region of the first strand. This region serves as the priming site for amplification. For blunt-ended polynucleotide fragments, the adapters are simply ligated to the blunt ends. For polynucleotide fragments with cohesive ends, the adapters are annealed to the 5′ or 3′ extensions of each polynucleotide fragment. Thus, one strand of each adapter also contains a second region that is substantially complementary to a region in the extensions of the polynucleotide fragments. Adapters useful in ligation-mediated amplification are typically designed so that contact with a ligase results in ligation of only one strand to each end of the polynucleotide fragments.

Conditions for annealing the adapter to the polynucleotide fragments, such as temperature, ionic strength, and oligonucleotide concentrations are generally selected to provide appropriate specificity of hybridization. Conditions suitable for annealing a given adapter to a particular 5′ or 3′ extension sequence are either known or can readily be determined by those skilled in the art.

The annealed adapters are contacted with a polynucleotide ligase, such as T4 polynucleotide ligase under suitable conditions, and for a sufficient time, to ligate an end of one strand of the adapters to an adjacent end of the polynucleotide fragment. This ligation is generally carried out according to standard techniques, i.e., in an appropriate ligation buffer including ATP. In ligation-mediated amplification, annealing of the adapters is performed by raising and then lowering the temperature of the mixture, followed by addition of ligase.

After ligation, the reaction mixture is generally denatured to remove the unligated adapter strand and the gap left is filled in by adding a suitable polymerase, such as Taq and/or Pwo, and dNTPs. The unligated adapter strand is then available for use as an amplification primer. As discussed in greater detail below, this primer can contain a functional group (such as an amino group) that facilitates immobilization of polynucleotides to a substrate. The sequences between the priming sites are amplified in a conventional amplification reaction. The selection of amplification protocols for various applications are well known to those of skill in the art. Guidance regarding various in vitro amplification methods can be found, for example, in Sambrook (1989) Molecular Cloning: A Laboratory Manual, 2nd Edition (Cold Spring Harbor Laboratory Press); U.S. Pat. No. 4,683,202 (issued in 1987 to Mullis et al.); PCR Protocols A Guide to Methods and Applications (Innis et al. eds) Academic Press Inc. San Diego, Calif. (1990); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science, 241: 1077-1080; Van Brunt (1990) Biotechnology, 8: 291-294; Wu and Wallace, (1989) Gene, 4: 560; and Barringer et al. (1990) Gene, 89: 117; as well as Smith, D. R. (1992) PCR Methods and Applications 2:21-27.

Preferably, the polymerase chain reaction (PCR) is used to amplify the polynucleotide fragments. For PCR, dNTPs, and one or more polymerases, such as Taq and/or Pwo polymerases, are added to the reaction mixture, which is then subjected to temperature cycling to allow repeated sequences of denaturation, primer annealing, and polynucleotide synthesis. An exemplary, preferred PCR amplification protocol is described in Example 1. This step produces an amplification product for each sample of polynucleotide fragments that is derived from a starting polynucleotide, such as a BAC clone. To fabricate an array containing 30,000 BAC clones, for example, each clone could be digested with a restriction enzyme and each of the resulting samples of polynucleotide fragments would be amplified to produce 30,000 amplification products.

If larger amounts of amplification products are desired, one or more additional rounds of amplification can be performed using the amplification products from the prior round of amplification as a template. An exemplary protocol including two rounds of amplification is described in Example 1. This feature of the method is particularly advantageous when preparing target solutions of polynucleotides from single-copy vectors, such as BACs, for which it is otherwise necessary to grow large cultures to obtain sufficient DNA for arraying.

Target Solutions

To form target solutions, the polynucleotide products of ligation-mediated amplification are isolated by any convenient method, such as, for example, precipitation by ethanol. Each polynucleotide product is resuspended to form a target solution suitable for application to a substrate. Suitable solutions should not significantly diminish the hybridization capacity of the polynucleotide products and should enable the polynucleotide products to adhere to the substrate.

Suitable solutions are well known to those of skill in the art and include, for example, 3×SSC and solutions containing one or more denaturants, such as formamide or dimethyl sulfoxide (e.g., 50% vol/vol DMSO in water). A 20% vol/vol DMSO solution is surprisingly better at solubilizing DNA than solutions containing more DMSO and is preferred. Target solutions intended for robotic spotting of microarrays preferably have a sufficiently low viscosity to allow spotting using conventional robotic techniques. In some embodiments, reproducible spotting of a precise amount of a target solution containing a predetermined amount of polynucleotides is desirable; however, differences in the amount of target solutions spotted can be normalized by including a control in the hybridization study, as is done, for example, in the technique of comparative genomic hybridization.

The concentration of the polynucleotide in the target solution should be high enough to allow detection of a hybridization signal from the corresponding target element of the array. Generally, good results are obtained using target solutions that have polynucleotide concentrations of about 0.2 μg/μl to about 2 μg/μl. Higher polynucleotide concentrations can be employed; however, improvements in signal level off at a polynucleotide concentration of about 1 μg/μl.

In one embodiment, the invention provides a collection of target solutions that is representative of a collection of YAC, BAC, P1, or PAC clones.

### Preparation of Polynucleotide Arrays

Application of Target Solutions to a Substrate

The target solutions of the invention can each be applied to a distinct location on a substrate to produce an array of polynucleotide-containing target elements. Substrates suitable for arraying polynucleotides are well-known and include, for example, a membrane, glass, quartz, or plastic. Exemplary membranes include nitrocellulose, nylon, diazotized membranes (paper or nylon), silicones, polyformaldehyde, cellulose, cellulose acetate, and the like. The use of membrane substrates (e.g., nitrocellulose, nylon, polypropylene) is advantageous because of well-developed technology employing manual and robotic methods of arraying targets at relatively high element densities. In addition, such membranes are generally available, and protocols and equipment for hybridization to membranes are well-known. Plastics suitable for use as array substrates include polyethylene, polypropylene, polystyrene, and the like. Other materials, such as ceramics, metals, metalloids, and semiconductive materials, can also be employed. In addition substances that form gels can be used. Such materials include proteins (e.g., gelatins), lipopolysaccharides, silicates, agarose and polyacrylamides. Where the substrate is porous, various pore sizes can be employed depending upon the nature of the system. Exemplary, preferred substrates include aminosilane, poly-lysine, and chromium substrates.

Substrates useful in the invention can have any convenient shape. Although the substrate typically has at least one flat, planar surface, substrates with non-planar surfaces are also within the scope of the invention. For example, the substrate can be made from beads, pins, or optical fibers.

Many methods for immobilizing polynucleotides on a variety of substrates are known in the art. The polynucleotide products described herein can be covalently or noncovalently bound to the substrate. The substrate surface can be prepared for immobilization using any of a variety of different materials, for example as laminates, depending on the desired properties of the array. Proteins (e.g., bovine serum albumin) or mixtures of macromolecules (e.g., Denhardt's solution) can be employed to avoid non-specific binding, simplify covalent conjugation, enhance signal detection or the like. If covalent bonding between a polynucleotide and the substrate surface is desired, the surface can be polyfunctional or capable of being polyfunctionalized. Functional groups useful for covalently bonding polynucleotides to substrate surfaces include carboxylic acids, aldehydes, amino groups, cyano groups, ethylenic groups, hydroxyl groups, mercapto groups, and the like. Alternatively, such functional groups can be introduced into the polynucleotide products of the invention. Methods for introducing various functional groups into polynucleotides are well-known and described, for example, in Bischoff et al., Anal. Biochem. (1987) 164:336-344; Kremsky et al., Nuc. Acids Res. (1987) 15:2891-2910. Nucleotides bearing functional groups can also added to the products of the ligation-mediated amplification method described above using PCR primers containing a modified nucleotide, or by enzymatic end-labeling with modified nucleotides. In a preferred embodiment, polynucleotide products according to the invention bear a functional group, such as, for example, an amino group.

The target solutions of the invention are applied to the substrate surface using any method that substantially maintains the hybridization capacity of the target solution polynucleotides. For fabrication of microarrays, the target solutions are applied by robotic spotting using a device such as that described in U.S. Pat. No. 5,807,522 (issued Sep. 15, 1998 to Brown and Shalon). The target solutions can be applied, for example, by tapping a capillary dispenser containing target solution against the substrate surface. To form a microarray, the average volume of each target solution applied to the substrate is less than about 2 nanoliters. Generally, at least about 0.002 nanoliters of each target solution is applied to the substrate. Preferably, between about 0.02 nanoliters and about 0.2 nanoliters of each target solution is applied.

A “print head” containing multiple, closely spaced dispensers or “printing tips” can be employed to facilitate array manufacture and to minimize the physical size of arrays, thereby reducing the amounts of polynucleotides required for each hybridization analysis. An exemplary system for fabricating a microarray by robotic spotting is described in Example 2.

Arrays

Arrays prepared according to the methods of the invention have target elements containing polynucleotides that are each representative of the polynucleotide from which the corresponding target element polynucleotides are derived (i.e, by amplification). In one embodiment, the invention provides an array in which each target element is representative of a YAC, BAC, P1 and/or PAC clone.

An array according to the invention can include target elements of any dimensions suitable for the intended application. Small target elements containing small amounts of concentrated target polynucleotides are conveniently used when the probe that is hybridized to them contains high complexity polynucleotides, since the total amount of probe available for binding to each target element during hybridization to the array will be limited. Such target elements also provide a hybridization signal that is highly localized and bright. Thus, target elements of less than about 1 cm in diameter are generally preferred. Exemplary target element sizes range from 1 μm to about 3 mm, and are preferably between about 5 μm and about 1 mm.

Target element density depends upon a number of factors, such as the substrate, the technique for applying target solutions to the substrate, the nature of the label to be hybridized to the array, and the like. Microarrays have target element densities of at least 100 target elements per cm² of substrate. Preferred microarrays have target element densities of at least 10³, 10⁴, 10⁵, and 10⁶ target elements per cm² of substrate.

All publications cited herein are hereby expressly incorporated by reference.

This invention is farther illustrated by the following specific, but non-limiting, examples. Procedures that are constructively reduced to practice are described in the present tense, and procedures that have been carried out in the laboratory are set forth in the past tense.

### EXAMPLE 1

### Preparation of Target Solutions from BAC Clones by Ligation-Mediated PCR

This study addressed the problems of the continual need to grow BACs for DNA and the problems with viscosity in printing BAC DNA by generating a PCR representation of the BAC. Ligation-mediated PCR was used to produce large amounts of BAC DNA that could be used to make low-viscosity target solutions suitable for robotic spotting. In this procedure, the DNA was first digested with MseI, an enzyme with a 4-base recognition site to maximize the frequency at which the DNA is cut. An adapter was then ligated to the digested DNA and used to prime an initial PCR amplification. To make DNA for spotting, a second PCR amplification was performed using the first PCR product as template.

DNA Isolation and Restriction Enzyme Digest

Cultures of BAC clones from the RP11 human BAC library were prepared by inoculating 5 μl LB with 1 μl from individual glycerol stocks and allowed to grow overnight. The overnight cultures were maintained at 4° C. for 8 hrs prior to use. Then, 25 mL cultures were prepared by inoculating LB medium with 200 μl of each overnight culture. These cultures were incubated at 37° C. in a shaking incubator for 14-16 hr (OD₆₀₀=0.25-0.35). BAC DNA was isolated from the cultures by standard alkaline lysis followed by purification over Qiagen Mini™ columns. Buffer volumes were increased as recommended by the manufacturer and routine yields were approximately 5 μg of DNA/25 ml culture. The DNA was minimally contaminated by the host bacterial genomic DNA (˜6%, based on number of E. coli sequence reads from a shotgun library prepared from the BAC DNA).

Isolated BAC DNA (20 ng to 300 ng) was digested with MseI in a 5 μl reaction mixture containing 1.5 μl DNA, 0.2 μl 10×One-Phor-All-Buffer-Plus™ (Pharmacia), and 1 μl MseI (New England Biolabs; diluted to 2 units/μl in 10×One-Phor-All-Buffer-Plus™). After incubation at 37° C. overnight, the DNA was diluted to a final concentration of 1 ng/μl in water.

Ligation-Mediated PCR

Adapter (primer 1), 5′-AGT GGG ATT CCG CAT GCT AGT-3′ (SEQ ID NO:1); containing a 5′ aminolinker and primer oligonucleotide (primer 2), 5′ TAA CTA GCA TGC-3′ (SEQ ID NO:2) was annealed to the TA overhangs that were created by digestion of the DNA with MseI by incubating 1 μl of the MseI digest product (1 ng/μl) with 0.5 μl of each primer (100 μM), 0.5 μl of 10×One-Phor-All-Buffer-Plus™ (Pharmacia) and 5.5 μl of H₂O. Annealing was initiated at 65° C. for 1 min. to inactivate the restriction enzyme, and then the temperature was lowered to 15° C., with a ramp of 1.3° C./min. Once the temperature reached 15° C., 1 μl ATP (10 mM) and 1 μl T4 DNA ligase (5 units/μl, Boehringer Mannheim) was added. The mixture was then incubated overnight.

Primary PCR was carried out as follows. 3 μl of 10×PCR buffer (Boehringer Mannheim, Expand Long Template™, buffer 1), 2 μl of dNTP's (10 mM), and 35 μl of water was added. The temperature was raised to 68° C. for 4 min to remove primer 2, and then a fill-in-reaction was carried out for 3 min after addition of 1 μl (3.5 units) of a mixture of Taq and Pwo DNA polymerases (Boehringer Mannheim, Expand Long Template™). Thermal cycling was carried out in a Perkin-Elmer Gene Amp PCR™ system 9700 block for 14 cycles of 94° C. for 40 sec, 57° C. for 30 sec, and 68° C. for 75 sec; followed by 34 cycles of 94° C. for 40 sec, 57° C. for 30 sec, 68° C. for 105 sec; and a final cycle of 94° C. for 40 sec, 57° C. for 30 sec and 68° C. for 5 min.

To make DNA for spotting, 1 μl of DNA from this primary PCR (approximately 100 ng/μl) was re-amplified in a 100 μl reaction containing 4 μM primer 1, 1×TAQ-buffer II™ (Perkin Elmer), 0.2 mM dNTP mix (Boehringer Mannheim), 5.5 mM MgCl₂ (Perkin Elmer), and 2.5 units Amplitaq Gold™ (5 units/μl, Perkin Elmer). The polymerase was activated by incubation at 95° C. for 10 min in a Perkin-Elmer Gene Amp™ PCR system 9700 block, and then thermal cycling was carried out for 45 cycles of denaturation at 95° C. for 30 sec, annealing at 50° C. for 30 sec, and polymerization at 72° C. for 2 min., followed by a final extension at 72° C. for 7 min.

Preparation of Target Solutions

The volume of each amplification reaction (containing ˜10 μg DNA/100 μl) was reduced to ˜50 μl by incubation in a fan oven (Techne Hybridizer HB-1D) at 45° C. for 75 min. The DNA was precipitated by addition of 2.5 volumes of ethanol and one-tenth volume of 3M sodium acetate. The solution was mixed and then centrifuged at 4,000 rpm for 75 min. The supernatant was removed and the pellet washed with 70% ethanol and then centrifuged again at 4,000 rpm for 45 min. The supernatant was removed, and the pellet was allowed to air dry. The DNA was then resuspended in 5 μl of 20% vol/vol DMSO in water.

Using this procedure, as many as 10,000 aliquots of spotting solution could be prepared from 100 ng of BAC DNA.

### EXAMPLE 2

### Arraying of Target Solutions

Target solutions were printed on a substrate using a print head with multiple, closely-spaced printing tips. The printing tips were dipped into target solutions in 864-well microtiter plates, which permitted spacing the pins on 3 mm centers. The print head contained 16 pins (in a 4×4 arrangement) that produces 12 mm×12 mm arrays. Target elements were printed on approximately 150 μm centers.

The printing pins were made from quartz capillary tubes that were tapered toward the tip. A typical design had a 75 μm inside diameter tube that narrowed to a 25-50 μm opening at the tip. The pins were individually spring-mounted in the print head so that the pins could move independently. Each was connected by flexible tubing to a manifold that supplied pressure or vacuum as required. Each print cycle began with cleaning the pins by drawing cleaning solutions through them under vacuum. They were then dried in an air blast and dipped into the microtiter plate. A slight vacuum was applied to draw target solutions into the pins. The print head was then moved along a gantry to a firm stop that precisely referenced its position. The array substrates were mounted on a precision X-Y stage and moved under the print head to the proper position, and the head was lowered for printing. Replicate target elements were printed for each target polynucleotide to allow averaging of hybridization signal across the replicates. 96 full genomic arrays containing triplicate copies of each of 3000 clones (1 Mb resolution in a mammalian genome), could be printed in 6-7 hours.

The above procedure was carried out using a variety of substrates, including aminosilane, poly-lysine, and chromium.

After spotting, the arrays were typically dried overnight (although this is not necessary) and then placed in a UV Stratolinker 2400™ (Stratagene) and treated twice with 65 mJoules to improve attachment of the DNA to the substrate.

Results

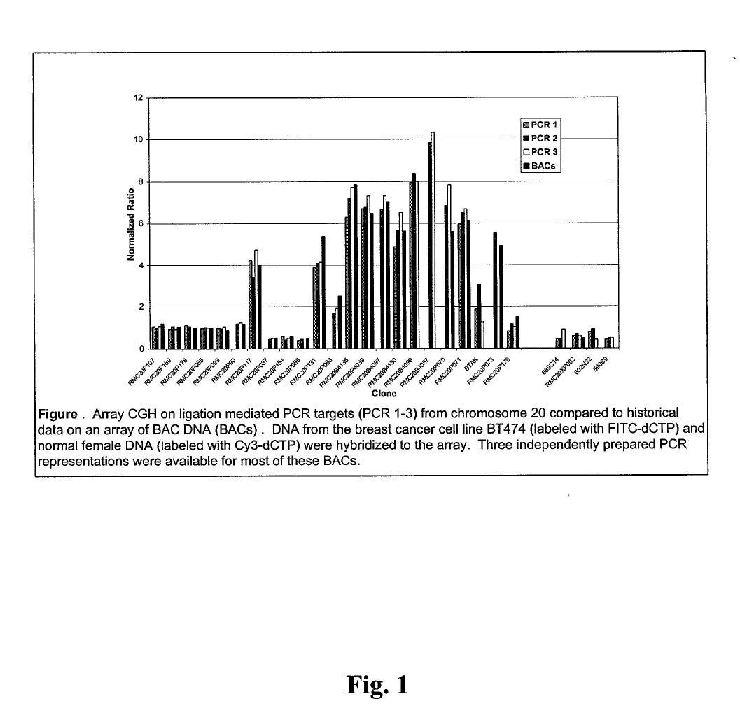

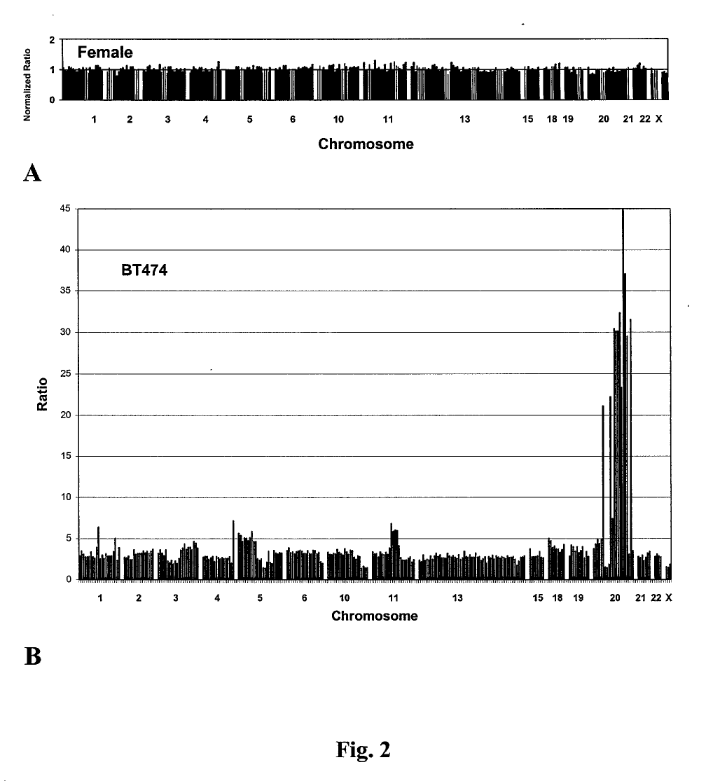

Side-by-side hybridization of arrayed BAC DNA and DNA prepared from the same BACs by ligation-mediated PCR yielded the same results (see FIG. 1), indicating that the DNA prepared by ligation-mediated PCR was representative of the starting BAC DNA. FIG. 2 shows the results of CGH to genome scanning array containing DNA from 400 BAC clones prepared by ligation-mediated PCR and arrayed as described in this example. FIG. 2 demonstrates that the methods described herein produce arrays that are representative of the starting polynucleotides.

What is claimed is:

1. A method for preparing amplification products useful for forming an array of polynucleotides that is representative of a plurality of first polynucleotides comprising:
a) providing a plurality of samples of double-stranded polynucleotide fragments, wherein each sample is derived from a first polynucleotide;
b) ligating adapters to each end of the polynucleotide fragments to produce modified polynucleotide fragments, wherein each adapter comprises a first strand and a second strand, the second strand having a region of substantial complementarity to a region of the first strand;
c) amplifying the modified polynucleotide fragments to produce an amplification product for each sample of polynucleotide fragments;
d) isolating each amplification product; and
e) resuspending each amplification product to form a target solution suitable for application to a substrate to produce an array of polynucleotides.

2. The method of claim 1 additionally comprising applying the target solutions to one or more substrates, wherein each target solution is applied to a distinct location on one substrate and/or target solutions are applied to different substrates that are combined to produce an array of polynucleotides.

3. The method of claim 1 wherein the double-stranded polynucleotide fragments are derived from a polynucleotide library.

4. The method of claim 3 wherein the polynucleotide library is a genomic DNA library.

5. The method of claim 3 wherein the polynucleotide library is a cDNA library.

6. The method of claim 3 wherein the double-stranded polynucleotide fragments are derived from YAC, BAC, P1 or PAC clones.

7. The method of claim 1 wherein the first polynucleotides each have a complexity of at least about 50 kilobases.

8. The method of claim 1 wherein the first polynucleotides each have a complexity of at least about 100 kilobases.

9. The method of claim 7 wherein the first polynucleotides each have a complexity of less than about 500 kilobases.

10. The method of claim 1 wherein the double-stranded polynucleotide fragments are obtained using one or more restriction endonucleases.

11. The method of claim 1 wherein the average length of the double-stranded polynucleotide fragments is less than about 5 kilobases.

12. The method of claim 11 wherein the average length of the double-stranded polynucleotide fragments is less than about 2 kilobases.

13. The method of claim 11 wherein the average length of the double-stranded polynucleotide fragments is greater than about 100 basepairs.

14. The method of claim 2 wherein the average volume of each target solution applied to the substrate is less than about 2 nanoliters.

15. The method of claim 14 wherein the average volume of each target solution applied to the substrate is equal to greater than about 0.002 nanoliters.

16. The method of claim 2 wherein the array comprises at least 1000 amplification products in a 1 cm² region of substrate.

17. The method of claim 2 wherein the target solutions are robotically spotted on the substrate.

18. The method of claim 2 wherein at least one strand of the adapters includes an amino group.

19. The method of claim 1 wherein the target solutions comprise dimethyl sulfoxide at a concentration of about 20% by volume.

20. An array of polynucleotides that is representative of a plurality of first polynucleotides wherein said array is produced according to the method of claim 2 and comprises at least 1000 amplification products in a 1 cm² region of substrate.

21. A plurality of target solutions prepared according to the method of claim 3.

22. The plurality of target solutions of claim 21 wherein the target solutions comprise dimethyl sulfoxide at a concentration of about 20% by volume.
